(19) United States
(12) Patent Application Publication
(10) Pub. No.: US 2026/0064857 A1
(43) Pub. Date: Mar. 5, 2026

(54) DNAZYME-ASSISTED DNA CRYPTOGRAPHY

(71) Applicant: Seagate Technology LLC, Fremont, CA (US)

(72) Inventors: Mengdi BAO, Shakopee, MN (US); Yasaman KESHTKARJAHROMI, Maple Grove, MN (US); Sriram CHARI, Shakopee, MN (US); Gemma MENDONSA, Edina, MN (US); Anil J. REDDY, Minneapolis, MN (US)

(21) Appl. No.: 18/817,104

(22) Filed: Aug. 27, 2024

(51) Int. Cl. G06F 21/60; C12P 19/34

(52) U.S. Cl. CPC G06F 21/602; C12P 19/34

(57) ABSTRACT

A sender may provide, to a recipient, encrypted DNA material comprising a set of DNA fragments. The sender may provide, to the recipient, a decryption key including instructions for ligating the set of DNA fragments to construct an encoded DNA strand having a nucleotide sequence that encodes a message.

### BACKGROUND

There is always a desire for more data storage and increased speed of writing to, and reading from that storage, as well as a desire for reduced cost for the stored data.

DNA is an emerging technology for data storage. DNA enables a large amount of data to be stored in a small volume. In certain DNA-based storage methods, DNA is synthesized using oligonucleotides (“oligos”). Oligos are prefabricated, synthesized DNA strands that are stored in reservoirs. The nucleotides (e.g., A, C, G. T; where “A” refers to adenine, “C” refers to cytosine, “G” refers to guanine, and “T” refers to thymine) of the synthesized DNA strand represent the encoded data.

### SUMMARY

This disclosure is directed to encoding data in DNA strands and encrypting DNA strands using one or more of cleavage or ligation processes.

In some aspects, the techniques described herein relate to a method, including: providing, to a recipient, encrypted DNA material including a set of DNA fragments; and providing, to the recipient, a decryption key including instructions for ligating the set of DNA fragments to construct an encoded DNA strand having a nucleotide sequence that encodes a message.

In some aspects, the techniques described herein relate to a method, including: synthesizing an encoded DNA strand having a nucleotide sequence that encodes a message by at least ligating a set of DNA fragments of encrypted DNA material; and decoding the nucleotide sequence to determine the message.

In some aspects, the techniques described herein relate to a method, including: synthesizing an encoded DNA strand, wherein a nucleotide sequence of the encoded DNA strand encodes a message; cleaving the encoded DNA strand into encrypted DNA material including a set of DNA fragments; storing a decryption key including instructions for ligating the set of DNA fragments to reconstruct the encoded DNA strand having the nucleotide sequence; accessing the decryption key; synthesizing the encoded DNA strand having the nucleotide sequence by at least ligating the set of DNA fragments of the encrypted DNA material; and decoding the nucleotide sequence to read the message.

Other systems and methods are also described herein.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. These and various other features and advantages will be apparent from a reading of the following detailed description.

### BRIEF DESCRIPTION OF THE DRAWING

The described technology is best understood from the following Detailed Description describing various implementations read in connection with the accompanying drawing.

FIG. 1A is a schematic rendering of a first DNA fragment.

FIG. 1B is a schematic rendering of a second DNA fragment.

FIG. 1C is a schematic rendering of a DNAzyme.

FIG. 2 is a schematic rendering of a ligation DNAzyme linking two DNA fragments.

FIG. 3 is a schematic rendering of a DNA strand formed from the ligation DNAzyme linking two DNA fragments of FIG. 2.

FIG. 4 is a schematic rendering of a DNAzyme.

FIG. 5A illustrates an example DNA strand that can be split into fragments using a cleavage DNAzyme.

FIG. 5B illustrates DNA fragments formed as a result of cleaving of the DNA strand of FIG. 5A using the cleavage DNAzyme of FIG. 5A.

FIG. 6 illustrates an example encryption scheme including generating encrypted DNA material by cleaving an encoded DNA strand into fragments using a cleavage encryption key.

FIG. 7 illustrates decrypting encrypted DNA material according to the encryption scheme described in FIG. 6 by reconstructing an encoded DNA strand from DNA fragments of the encrypted DNA material using a ligation decryption key.

FIG. 8 illustrates an example encryption scheme including generating an encrypted DNA strand from an encoded DNA strand using a cleavage encryption key and a ligation encryption key.

FIG. 9 illustrates an example of decrypting an encrypted DNA strand according to the encryption scheme described in FIG. 8 using a cleavage decryption key and a ligation decryption key.

FIG. 10 depicts a process for encrypting DNA strands that encode messages.

FIG. 11 illustrates an example computing device for use in implementing the described technology.

### DETAILED DESCRIPTION

DNA cryptography offers advantages in high-density storage, durability, security, and versatility compared to traditional cryptographic approaches. Conventional DNA cryptography involves encoding (e.g., via binary encoding or other encoding scheme) a message into a DNA sequence, encrypting the DNA sequence using an encryption method (e.g., an encryption key) to determine an encrypted DNA sequence, and synthesizing an encrypted DNA strand that includes the encrypted DNA sequence. The (synthesized) encrypted DNA strand may be stored in a protective environment until it is desired to be read. The encrypted DNA strand is then sequenced to determine the encrypted DNA sequence, the encrypted DNA sequence is decrypted using a decryption method (e.g., a decryption key) to determine the original DNA sequence into which the message was originally encoded, and the message is read from the decrypted DNA sequence. However, such conventional DNA methods involve synthesis of DNA strands having sequences that are pre-encrypted. Accordingly, such methods are insecure because a bad actor having expertise in or having access to DNA sequencing methods can easily determine the encrypted DNA sequence from the encrypted DNA strand and then attempt to decrypt (e.g., with the assistance of a computer) the encrypted DNA sequence and read the encoded message.

The described technology addresses the deficiencies of the conventional encryption methods used in DNA-based storage schemes. The encryption schemes of the described technology involve one or more of ligation or cleavage of an encoded DNA strand that encodes a message to generate encrypted DNA material (e.g., fragments of the encoded DNA strand or an encrypted DNA strand formed of rearranged sections of the encoded DNA strand). As a result, to read the encrypted DNA strand generated using the described technology, a reader of the message must first know how to perform one or more specific ligation or cleavage processes to reconstruct, from the encoded DNA material, the encoded DNA strand before sequencing the encoded DNA strand to determine the message. Accordingly, the complexity of encryption in the disclosed technology is greater than conventional encryption techniques because it incorporates physical encryption techniques (e.g., ligation and/or cleavage chemical reactions) of an encoded DNA strand that are not present in conventional encryption schemes.

In the following description, reference is made to the accompanying drawings that form a part hereof and which is shown by way of illustration of at least one specific implementation. The following description provides additional specific implementations. It is to be understood that other implementations are contemplated and may be made without departing from the scope or spirit of the present disclosure. The following detailed description, therefore, is not to be taken in a limiting sense. While the present disclosure is not so limited, an appreciation of various aspects of the disclosure will be gained through a discussion of the examples, including the figures, provided below. In some instances, a reference numeral may have an associated sub-label consisting of a lower-case letter to denote one of multiple similar components. When reference is made to a reference numeral without specification of a sub-label, the reference is intended to refer to all such multiple similar components.

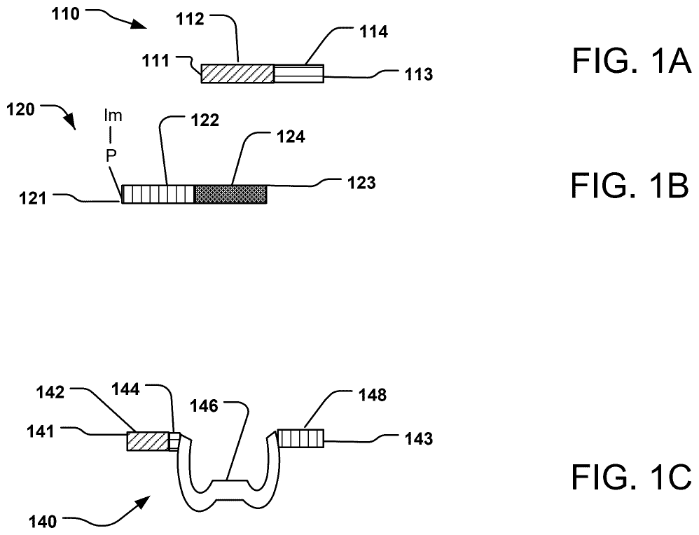

FIGS. 1A through IC show examples of the components for forming a DNA strand or gene using a ligation DNAzyme.

In FIG. 1A a first DNA fragment 110. This first DNA fragment 110 is shown with a first sequence subsection 112 at a first end 111 and a second sequence subsection 114 at a second end 113, each of the subsections 112, 114 composed of a plurality of nucleotides.

FIG. 1B shows a second DNA fragment 120. This second DNA fragment 120 is shown with a first sequence subsection 122 at a first end 121 and a second sequence subsection 124 at a second end 123, each of the subsections 122 and 124 composed of a plurality of nucleotides. The first sequence subsection 122 at the first end 121 is an S1 end, and the second sequence subsection 124 at the second end is an S2 end. Additionally, the first sequence subsection 122 is shown with a phosphate-imidazole group, a conventional feature when using certain ligation DNAzymes for synthesis.

The second DNA fragment 120 may be composed of a number (e.g., four, five, eight, twelve, or other number) of nucleotides forming the subsection 122 and the subsection 124. In some implementations, the subsections 122 and 124 are the beginning subsections of a longer DNA strand. These S1 and S2 linking subsections 122 and 124 may have any number of nucleotides.

The first DNA fragment 110 and the second DNA fragment 120 may, in some implementations, each be composed of six to 20 nucleotides, with the end nucleotides (e.g., linking subsections 114 and 122) complementary to ends of a ligation DNAzyme, discussed below. In some embodiments, each of the linking subsections 114 and 122 will have a number (e.g., four, five, six, eight, ten, twelve, or other number) of nucleotides corresponding to ends of a ligation DNAzyme.

FIG. 1C shows a ligation DNAzyme 140. The ligation DNAzyme 140 has four sequence sections, a first sequence section 142 at a first end 141 of the DNAzyme 140, a second sequence section 144, a third sequence section 146, and a fourth sequence section 148 at the second end 143 of the DNAzyme 140, each of the sections 142, 144, 146, 148 composed of a plurality of nucleotides. The section 146 of the DNAzyme 140 is the E47 sequence whereas the sections 142, 144, and 148 are tailored to the particular application. The sequence section 148 at the second end 143 is complimentary to an S1 end. The ligation DNAzyme 140 described herein may be used for the synthesis of the DNA fragment components (e.g., the first DNA fragment 110 and the second DNA fragment 120) and/or synthesis of longer DNA strands (e.g., a first DNA strand terminating with a sequence corresponding to the first DNA fragment 110 and a second DNA strand commencing with a sequence corresponding to the second DNA fragment 120). However, other methods (e.g., enzyme-based methods other than DNAzymes) may be used instead of or in addition to using DNAzymes.

Together, the first DNA fragment 110 (e.g., or first DNA strand terminating with a nucleotide sequence corresponding to the sequence of the first DNA fragment 110), the second DNA fragment 120 (e.g., or second DNA strand commencing with a nucleotide sequence corresponding to the sequence of the second DNA fragment 120), the ligation DNAzyme 140, or other molecule/enzyme used for DNA ligation (e.g., synthesis), are part of a system that can be used to form a DNA strand or gene from two component DNA strands or genes. In some implementations, the DNA fragments (e.g., the first DNA fragment 110, the second DNA fragment 120) are part of a library of DNA fragments and the ligation DNAzyme 140 is part of a library of ligation DNAzymes. Each of the libraries is composed of multiple (e.g., hundreds, thousands) of DNA fragments (e.g., or commencing/terminating sequences of DNA strands that may be joined) and ligation DNAzymes modified to ligate with the DNA fragments/sections.

Although the first DNA fragment 110 and the second DNA fragment 120 are shown with subsections 112, 114, and 122, 124, respectively, it is to be understood that additional or fewer subsections may be present in one or both of the first DNA fragment 110 and the second DNA fragment 120. The example ligation DNAzyme 140 has at least three sections, with one of the sections being the catalytic portion, e.g., E47.

The different patterns in the sequence sections illustrated in FIG. 1A-IC designate different complementary sequences, for example, those that will ligate, or join.

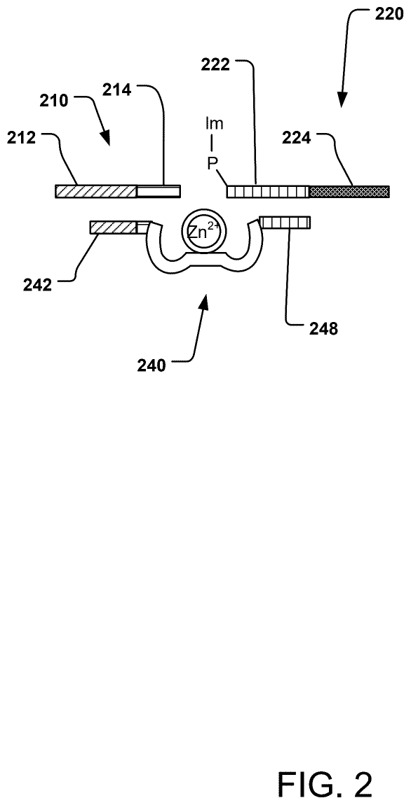

FIG. 2 shows the first DNA fragment of FIG. 1A and the second DNA fragment of FIG. 1B ligated using the ligation DNAzyme of FIG. 1C, in a particular order based on the sequence sections.

In FIG. 2, the first subsection 214 of the first DNA fragment 210 is joined to the S2 first subsection of the ligation DNAzyme 240; particularly, the sequence subsection 212 is complementary to and thus ligates with the sequence subsection 242 and the sequence subsection 214 is complementary to and ligates with the subsection 242. At the S1 second end subsection of the ligation DNAzyme 240, the sequence subsection 248 is complementary to and ligates with the subsection 222 of the second DNA fragment 220 (which includes subsection 222, subsection 224) at the S1 first end subsection 212.

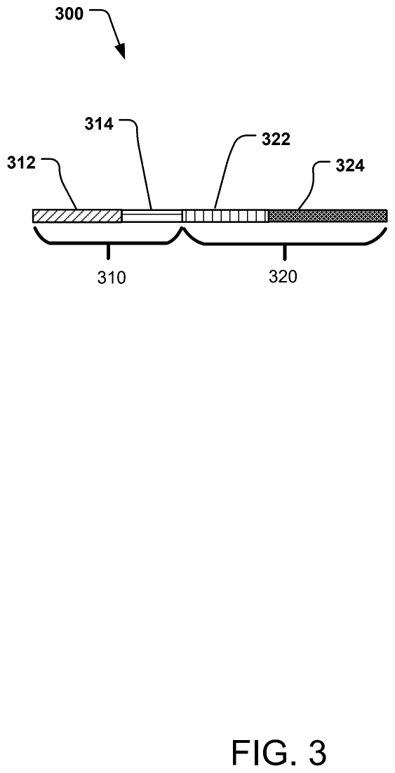

Accordingly, the ligation DNAzymes 240 may be used to attach the first DNA fragment 210 and the second DNA fragment 220. In such a manner, a DNA strand 300, shown in FIG. 3, is formed from the first DNA fragment 310 (including the subsection 312 and the subsection 314) and the second DNA fragment 320 (e.g., including the subsection 322 and the subsection 324). As indicated above, the 3′ end of the second DNA fragment 320 is an ‘activated’ end, activated by phosphate and imidazole before ligation. In some implementations, during ligation, the phosphate and imidazole release and do not appear in the DNA strand 300. In some implementations, the ligation DNAzyme is removed by various means, e.g., chemical, or physical methods that can include heat, strand displacement, or conjugation to magnetic beads.

The DNA strand 300, as formed above, may be faster and less expensive to form than DNA strands ligated using enzymes. By replacing enzymes with ligation DNAzymes, the cost of forming large DNA strands or otherwise joining component DNA strands into a single DNA strand for data storage is greatly reduced. Using ligation DNAzymes also increases the flexibility available during the assembly method. As shown above, ligation DNAzymes can be used to attach component DNA strands/fragments, eliminating the enzymes which can be the most expensive step. Additionally, ligation DNAzymes can be used to assemble larger DNA strands to form DNA strands or genes having sufficient length to encode usable amounts of data.

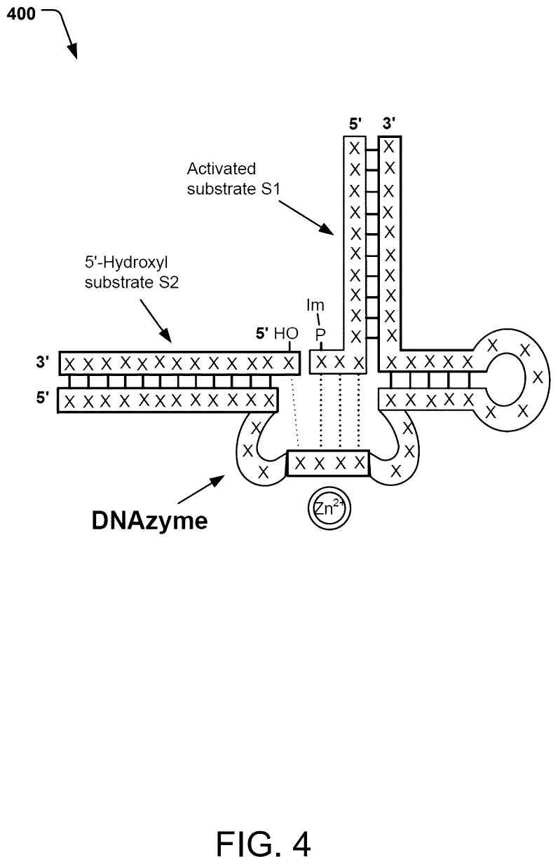

FIG. 4 illustrates an example ligation DNAzyme 400, such as an E47 DNAzyme, as it ligates or joins two DNA fragment strands, referred to herein as S1 and S2, where S1 is a 3′-phosphate-imidazole activated substrate and S2 is a S′-hydroxyl substrate; this ligation occurs in the presence of zinc or copper ions, with zinc being shown in FIG. 4. It is noted that all of the S1 DNA fragment, the S2 DNA fragment and the ligation DNAzyme 400 are represented with generic nucleotides designated as “X”; it is understood that in actuality these X designations will be a nucleotide A, C, T, G. The ligation DNAzyme catalyst molecule, e.g., E47, has a folded portion with a fixed sequence and structure; the 5′ and 3′ arms of the ligation DNAzyme 400, however, tolerate modifications to the sequence. Any or all the ligation DNAzyme 400, the 5′ end of the S1 DNA fragment or the 3′ end of the S2 DNA fragment can be modified to allow the S1 DNA fragment to join to the ligation DNAzyme 400 and the S2 DNA fragment to join to the ligation DNAzyme 400. This ligation methodology is used to join component DNA stands that can be used for data encoding.

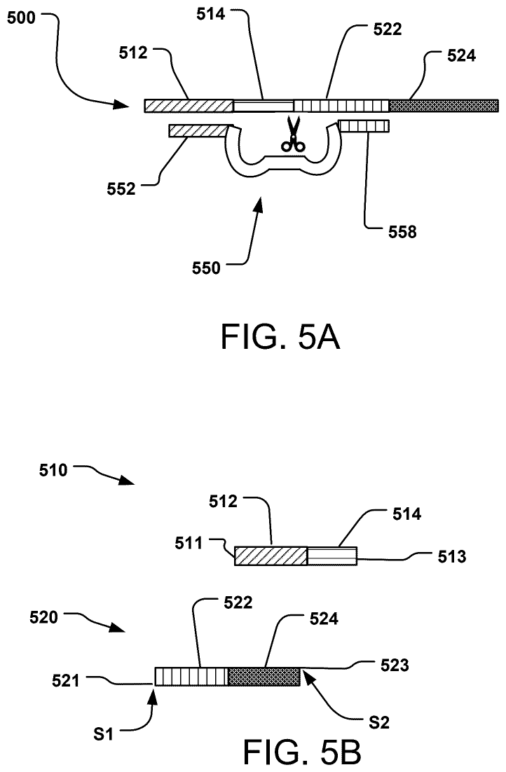

FIG. 5A illustrates an example DNA strand 500 that can be split into fragments using a cleavage DNAzyme 550. For example, the DNA strand 500 is the same as the example DNA strand 300. Cleavage DNAzymes (e.g., the cleavage DNAzyme 550) may be used to cleave (e.g., separate, split) the DNA strand 500 at a specific point (e.g., indicated by the scissors icon in FIG. 5A) in the sequence of the DNA strand 500 based on how subsections of the cleavage DNAzyme 550 binds to subsections of the DNA strand 500. For example, the sequence subsection 512 of the DNA strand 500 is complementary to and thus hybridizes with the sequence subsection 552 of the cleavage DNAzyme 550 and the sequence subsection 522 of the DNA strand 500 is complementary to and hybridizes with the subsection 558 of the cleavage DNAzyme 550.

FIG. 5B illustrates DNA fragments 510 and 520 formed as a result of leaving of the DNA strand 500 of FIG. 5A using the cleavage DNAzyme 550 of FIG. SA. The first DNA fragment 510 has a first sequence subsection 512 at a first end 511 and a second sequence subsection 514 at a second end 513, each of the subsections 512, 514 composed of a plurality of nucleotides. The second DNA fragment 520 has a first sequence subsection 522 at a first end 521 (S1) and a second sequence subsection 524 at a second end 523 (S2), each of the subsections 522 and 524 composed of a plurality of nucleotides. The first sequence subsection 522 at the first end 521 is an S1 end, as indicated in FIG. 5B, and the second sequence subsection 524 at the second end is an S2 end, as indicated in FIG. 5B. In some implementations, metal ions (such as Zn2+, Mg2+. Ca2+, etc.) are used to enable DNAzyme-based cleavage.

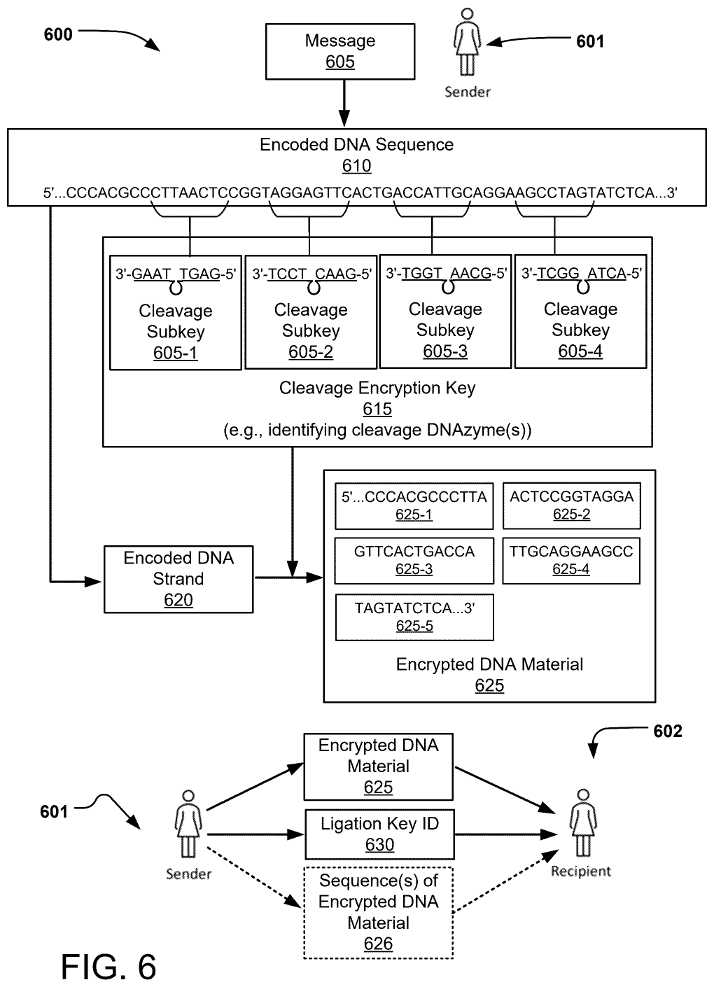

FIG. 6 illustrates an example encryption scheme 600 including generating encrypted DNA material 625 by cleaving an encoded DNA strand 620 into fragments using a cleavage encryption key 615. A sender 601 encodes a message 605 to generate an encoded DNA sequence 610 using a DNA encoding scheme. The message 605 may be one or more of a text, image data, video data, or other data. In some implementations, the sender 601 may encode and encrypt the message 605 according to the encryption scheme 600 for storage of the message 605 and later access of the message 605. In some implementations, the sender 601 may encode and encrypt the message 605 according to the encryption scheme 600 for transmission to and reading by a recipient 602. In some implementations, the sender 601 may use a computing device to assist in encoding the message 605 into the encoded DNA sequence 610.

The DNA encoding scheme used to encode the message 605 into the encoded DNA sequence 610 may be a binary encoding scheme, a ternary encoding scheme, a symbol-linker encoding scheme (e.g., motif-by-motif encoding scheme), or other encoding scheme in which data (e.g., text, symbols, numbers, other values, etc.) of the message 605 is represented by a nucleotide sequence. In the example encoded DNA sequence 610 depicted in FIG. 6, the nucleotide sequence encoding the message 605 is “ . . . . CCCACGCCCTTAACTCCGGTAGGAGTTCACTGACCATTGCAGGAAGCCTAGTATC TCA . . . ,” with the “ . . . . CCC” end being the 5 prime (5′) end of the encoded DNA sequence 610 and “ . . . . TCA” end being the 3 prime (3′) end of the encoded DNA sequence 610. As indicated by the use of ellipses on either end, the encoded DNA sequence 610 may be longer than the portion depicted that encodes the message 605.

The sender 601 generates (e.g., synthesizes) an encoded DNA strand 620 using the encoded DNA sequence 610. For example, the encoded DNA strand 620 is a physical strand of DNA that incorporates the nucleotide sequence of the encoded DNA sequence 610. One or more DNA synthesis methods may be used to generate the encoded DNA strand 620, for example, a nucleotide-by-nucleotide gene synthesis approach or via joining component subsections of the DNA strand 620 together to form the encoded DNA strand 620.

Using the encoded DNA strand 620, the sender 601 generates encrypted DNA material 625 from the encoded DNA strand 620 using one or more cleavage DNAzymes specified in a cleavage encryption key 615. For example, the cleavage encryption key 615 may include cleavage subkeys (e.g., cleavage subkey 605-1, cleavage subkey 605-2, cleavage subkey 605-3, cleavage subkey 605-4) that specify specific DNAzymes to be used to cleave the encoded DNA strand 620. The one or more cleavage DNAzymes cleave (e.g., split) the encoded DNA strand 620 at specific locations within its nucleotide sequence to generate a set of DNA fragments (e.g., DNA fragment 625-1, DNA fragment 625-2, DNA fragment 625-3, DNA fragment 625-4, DNA fragment 625-5).

For example, cleavage subkey 605-1 defines a cleavage DNAzyme having subsections GAAT and TGAG which are configured to hybridize with a corresponding CTTAACTC portion of the nucleotide sequence of the encoded DNA strand 620 and cleave into CTTA-ACTC, where “-” represents the cleavage. Cleavage subkey 605-2 defines a cleavage DNAzyme having subsections TCCT and CAAG which are configured to hybridize with a corresponding AGGAGTTC portion of the nucleotide sequence of the encoded DNA strand 620 and cleave into AGGA-GTTC, where “-” represents the cleavage. Cleavage subkey 605-3 defines a cleavage DNAzyme having subsections TGGT and AACG which are configured to hybridize with a corresponding ACCATTGC portion of the nucleotide sequence of the encoded DNA strand 620 and cleave into ACCA-TTGC, where “-” represents the cleavage. Cleavage subkey 605-4 defines a cleavage DNAzyme having subsections TCGG and ATCA which are configured to bind to a corresponding AGCCTAGT portion of the nucleotide sequence of the encoded DNA strand 620 and cleave into AGCC-TAGT, where “-” represents the cleavage. The cleavage subkeys and their corresponding cleavage DNAzymes illustrated in FIG. 6 are one example. Another number (e.g., more, or less) of cleavage DNAzymes may be used other than the example four cleavage DNAzymes illustrated in FIG. 4. In some implementations, a single cleavage DNAzyme may be used instead of multiple cleavage DNAzymes. Other DNAzymes may be used other than the four example DNAzymes specified by the example cleavage subkeys (e.g., cleavage subkey 605-1, cleavage subkey 605-2, cleavage subkey 605-3, cleavage subkey 605-4) illustrated in FIG. 6.

In the example illustrated in FIG. 6, cleaving the encoded DNA strand 620 using the DNAzymes specified in the cleavage encryption key 615 results in encrypted DNA material 625 that includes DNA fragment 625-1 having nucleotide sequence CCCACGCCCTTA, DNA fragment 625-2 having nucleotide sequence ACTCCGGTAGGA, DNA fragment 625-3 having nucleotide sequence GTTCACTGACCA, DNA fragment 625-4 having nucleotide sequence TTGCAGGAAGCC, and DNA fragment 625-4 having nucleotide sequence TAGTATCA.

The five DNA fragments illustrated in FIG. 6 are one example, and fewer or more fragments may be generated by using the cleavage DNAzyme(s) specified in the cleavage encryption key 615. The sender 601 may conduct appropriate chemical reactions to cause the cleavage DNAzyme(s) to cleave the encoded DNA strand 620 into the encoded DNA material 625 that includes the set of DNA fragments (e.g., DNA fragment 625-1, DNA fragment 625-2, DNA fragment 625-3, DNA fragment 625-4, DNA fragment 625-5). In some implementations, cleavage enzymes (e.g., Cas9, restriction enzymes, etc.) may be used instead of, or in addition to, cleavage DNAzymes. In some implementations, instead of cleaving the encoded DNA strand 620, the sender 602 may obtain the encoded DNA material by synthesizing the set of DNA fragments directly.

The described technology provides multiple ways for the sender 601 to share the encrypted DNA material 625 with the recipient 602. For example, the sender 601 may provide (e.g., by mailing, providing in person, leaving in a location accessible to the recipient 602, or otherwise providing or making available to the recipient 602) the encrypted DNA material 625 to the recipient 602.

In some implementations, instead of providing the encrypted DNA material 625 to the recipient, the sender 601 may provide sequence(s) of the encrypted DNA material 626 to the recipient 602. The sequence(s) of the encrypted DNA material 626 may include a sequence for each of the corresponding DNA fragments of the set of DNA fragments of the encrypted DNA material 625. The recipient, 602, in these implementations, then synthesizes the encrypted DNA material 625 (e.g., synthesis of the set of DNA fragments) using the sequence(s) of the encrypted DNA material 626 using one or more DNA synthesis schemes. In some implementations, the sender 601 transmits the sequence(s) of the encrypted DNA material 626 via one or more computing devices or otherwise communicates the sequence(s) of the encrypted DNA material 626 to the recipient 602 or a computing device accessible to the recipient 602. For example, the sender 601 transmits the sequence(s) of the encrypted DNA material 626 to the recipient 602 via email, text message, fax, voice message, video, a messaging application, or another method of communication.

In addition to either providing or otherwise making the encrypted DNA material 625 available to the recipient 602, either directly (e.g., providing the physical encrypted DNA material) or indirectly (e.g., providing the sequence(s) of the encrypted DNA material 626 for the recipient 602 to synthesize the encrypted DNA material 625), also provides a ligation key identifier (ID) 630 to the recipient 602. The ligation key ID 630 specifics one or more ligation DNAzymes that may be used by the recipient 602 to ligate the DNA fragments of the encrypted DNA material 625 in the proper order to recreate the encoded DNA strand 620. In some implementations, the sender 601 transmits the ligation key ID 630 via one or more computing devices and/or via one or more network connections or otherwise communicating the ligation key ID 630 to the recipient 602 or to a computing device accessible to the recipient 602. For example, the sender 601 transmits the ligation key ID 630 to the recipient 602 via email, text message, fax, voice message, video, messaging application, or other method of communication.

In some implementations, enzymes are used rather than DNAzymes. For example, anywhere cleavage DNAzymes are used, a CRISPR/Cas system may be used instead of or in addition to the cleavage DNAzymes. Anywhere ligation DNAzymes are used, a splint ligation process using T4 ligase may be used instead of or in addition to the ligation DNAzymes.

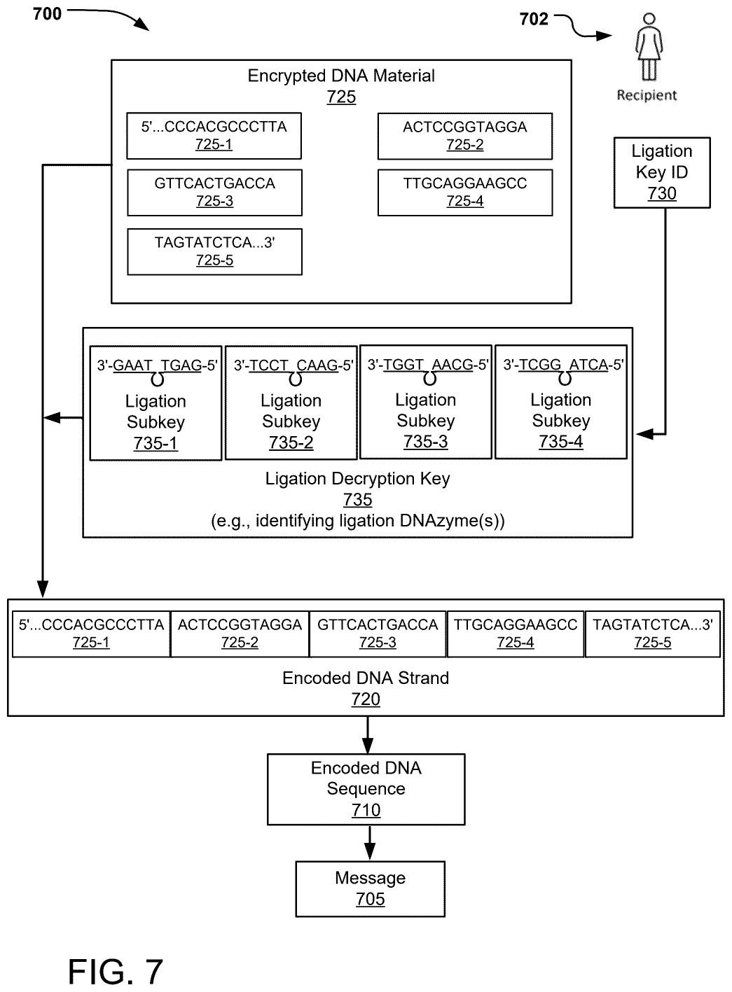

FIG. 7 illustrates an example 700 of decrypting encrypted DNA material according to the encryption scheme described in FIG. 6 by reconstructing an encoded DNA strand from DNA fragments of the encrypted DNA material using a ligation decryption key.

In some implementations, a recipient 702 receives encrypted DNA material 725 from a sender or otherwise accesses the encrypted DNA material 725. In some implementations, the recipient 702 synthesizes the encrypted DNA material 725 from the sequence(s) of the encrypted DNA material provided to the recipient 702 by the sender or otherwise accessed by the recipient 702. The recipient 702 receives (e.g., from the sender) or otherwise accesses a ligation key ID 730 associated with a ligation decryption key 735 that specifies one or more ligation DNAzymes for reconstructing the encoded DNA strand 720 from the DNA fragments of the encrypted DNA material 725.

The encrypted DNA material 725 includes a set of DNA fragments (e.g., DNA fragment 725-1 having nucleotide sequence CCCACGCCCTTA, DNA fragment 725-2 having nucleotide sequence ACTCCGGTAGGA, DNA fragment 725-3 having nucleotide sequence GTTCACTGACCA, DNA fragment 725-4 having nucleotide sequence TTGCAGGAAGCC, and DNA fragment 725-4 having nucleotide sequence TAGTATCA) of an encoded DNA strand 720 that encodes a message 705. In some implementations, the fragments were cleaved from the encoded DNA strand 720 using one or more cleavage DNAzymes specified in a cleavage encryption key (e.g., using the encryption scheme 600 illustrated in FIG. 6). In some implementations, the fragments are synthesized directly by the sender 701 or by the recipient 702 instead of being cleaved from the encoded DNA strand 720.

Using the encrypted DNA material 725, the recipient 702 reconstructs the encoded DNA strand 720 using one or more ligation DNAzymes specified in a ligation decryption key 735. The ligation decryption key 735 is associated with the ligation key ID 730 received from the sender or otherwise accessed by the recipient 702. For example, the ligation decryption key 735 may include ligation subkeys (e.g., ligation subkey 735-1, ligation subkey 735-2, ligation subkey 735-3, ligation subkey 735-4) that specify specific DNAzymes to be used to ligate the DNA fragments of the encrypted DNA material 725 in the correct order to reconstruct the encoded DNA strand 720. The one or more ligation DNAzymes ligate (e.g., join) ends of the DNA fragments.

For example, ligation subkey 735-1 defines a ligation DNAzyme having subsections GAAT and TGAG which are configured to bind to a corresponding CTTA portion of the fragment 725-1 and the ACTC portion of the DNA fragment 725-2 and to ligate the DNA fragment 725-1 and the DNA fragment 725-2. Ligation subkey 735-2 defines a ligation DNAzyme having subsections TCCT and CAAG which are configured to hybridize with a corresponding AGGA portion of the DNA fragment 725-2 and the GTTC portion of the DNA fragment 725-3, and to ligate the DNA fragment 725-2 and the DNA fragment 725-3. Ligation subkey 735-3 defines a ligation DNAzyme having subsections TGGT and AACG which are configured to hybridize with a corresponding ACCA portion of the DNA fragment 725-3 and the TTGC portion of the DNA fragment 725-4, and to ligate the DNA fragment 725-3 and the DNA fragment 725-4. Ligation subkey 735-4 defines a cleavage DNAzyme having subsections TCGG and ATCA which are configured to hybridize with a corresponding AGCC portion of the DNA fragment 725-4 and the TAGT portion of the DNA fragment 725-5, and to ligate the DNA fragment 725-4 and the DNA fragment 725-5.

The ligation subkeys and their corresponding ligation DNAzymes are illustrated in FIG. 7 is one example. Another number (e.g., more, or less) of ligation DNAzymes may be used other than the example four ligation DNAzymes illustrated in FIG. 7. In some implementations, ligation enzymes or chemical ligation methods may be used instead of or in addition to ligation DNAzymes.

In the example illustrated in FIG. 7, ligating the encrypted DNA material 725 using ligation DNAzymes specified in the ligation decryption key 735 results in the encoded DNA strand 720. FIG. 7 illustrates how the ligated fragments (e.g., DNA fragment 725-1. DNA fragment 725-2, DNA fragment 725-3, DNA fragment 725-4, DNA fragment 725-5) of the encrypted DNA material 725 form the encoded DNA strand 720.

The recipient 702 determines an encoded DNA sequence 710 of the encoded DNA strand 720, for example, using DNA sequencing techniques. For example, the encoded DNA sequence 710 includes the nucleotide sequence CCCACGCCCTTAACTCCGGTAGGAGTTCACTGACCATTGCAGGAAGCCTAGTATCTC A. The recipient 702 decodes the encoded DNA sequence 710 to determine the message 705 that was represented by the encrypted DNA material 725. The message 705 may be one or more of a text, image data, video data, or other data. In some implementations, the recipient 702 may decode the message 705 according to a binary encoding scheme, a ternary encoding scheme, a symbol-linker encoding scheme (e.g., motif-by-motif encoding scheme), or other encoding scheme in which data (e.g., text, symbols, numbers, other values, etc.) of the message 705 is determined from the nucleotide sequence of the encoded DNA sequence 710.

Using the encryption scheme of FIG. 6 and the decryption scheme of FIG. 7, if an entity other than the recipient 702 obtains the short fragments of the encrypted DNA material 725 (with the number of fragments being n), the probability of guessing the correct order of the fragments to reconstruct the encoded DNA sequence 710 is 1/n!. Although the entity other than the recipient 702 may obtain some information by sequencing the fragments, as the number of fragments increases, the likelihood of correctly guessing the order becomes very low.

In some implementations, enzymes are used rather than DNAzymes. For example, anywhere cleavage DNAzymes are used, a CRISPR/Cas system may be used instead of or in addition to the cleavage DNAzymes. Anywhere ligation DNAzymes are used, a splint ligation process using T4 ligase may be used instead of or in addition to the ligation DNAzymes.

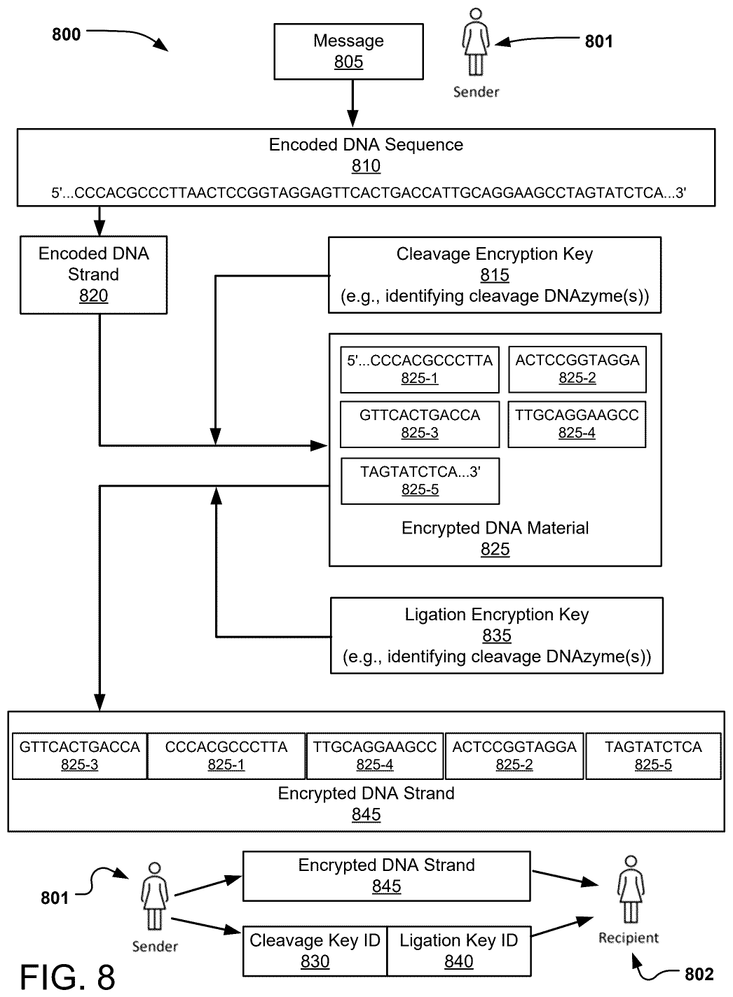

FIG. 8 illustrates an example encryption scheme 800 including generating an encrypted DNA strand 845 from an encoded DNA strand 820 using a cleavage encryption key 815 and a ligation encryption key 835. For example, a sender 801 encodes a message 805 into an encoded DNA sequence 810 and synthesizes an encoded DNA strand 820 that includes the encoded DNA sequence 810.

The encryption scheme 800 involves a cleavage process involving a cleavage encryption key 815 and a ligation process involving a ligation encryption key 835. In the cleavage process, the encoded DNA strand 820 is cleaved (e.g., by the sender 801) into encrypted DNA material 825 that includes a set of fragments (e.g., fragment 825-1, fragment 825-2, fragment 825-3, fragment 825-4, fragment 825-5) using cleavage DNAzymes specified in the cleavage encryption key 815. In some implementations, however, the set of fragments is synthesized directly by the sender 802 or by a recipient instead of being cleaved from the encoded DNA strand 820.

In the ligation process, the fragments (e.g., fragment 825-1, fragment 825-2, fragment 825-3, fragment 825-4, fragment 825-5) of the encrypted DNA material 825 are ligated (e.g., by the sender 801) into encrypted DNA strand 845 using ligation DNAzymes specified in the ligation encryption key 835. However, in some implementations, the encrypted DNA strand 845 can be synthesized directly by the sender 801 or by the recipient instead of being synthesized by cleaving the encoded DNA strand 820 into the fragments and then ligating the fragments.

As illustrated in FIG. 8, the order of the nucleotide sequence of the encrypted DNA strand 845 is different than the order of the nucleotide sequence of the encoded DNA strand 820 (e.g., which corresponds to the encoded DNA sequence 810). For example, the encrypted DNA strand 845 corresponds to the ligated fragments in the order of the fragment 825-3, followed by the fragment 825-1, followed by the fragment 825-4, followed by the fragment 825-2, followed by the fragment 825-5. However, the original encoded DNA strand 820, before its cleavage, corresponds to the fragment 825-1, followed by the fragment 825-2, followed by the fragment 825-3, followed by the fragment 825-4, followed by the fragment 825-5.

The described technology provides multiple ways for the sender 801 to share the encrypted DNA strand 845 with the recipient 802. For example, the sender 801 may provide (e.g., by mailing, providing in person, leaving in a location accessible to the recipient 802, or otherwise providing or making available to the recipient 802) the encrypted DNA strand 845 to the recipient 802.

In some implementations, instead of providing the encrypted DNA strand 845 to the recipient 802, the sender 801 may provide the nucleotide sequence of the encrypted DNA strand 845 to the recipient 802. The recipient, 802, in these implementations, then synthesizes the encrypted DNA strand 845 using one or more DNA synthesis schemes. In some implementations, the sender 801 transmits the sequence of the encrypted DNA strand 845 via one or more computing devices or otherwise communicates the sequence to the recipient 802 or to a computing device accessible to the recipient 802. For example, the sender 801 transmits the sequence to the recipient 802 via email, text message, fax, voice message, video, messaging application, or other method of communication.

In addition to either providing or otherwise making the encrypted DNA strand 845 available to the recipient 802, either directly (e.g., providing the physical encrypted DNA strand 845) or indirectly (e.g., providing the sequence of the encrypted DNA strand 845 for the recipient 802 to synthesize), also provides a cleavage key ID 830 and a ligation key ID 840 to the recipient 802. The cleavage key ID 830 specifies one or more cleavage DNAzymes that may be used by the recipient 802 to cleave the encrypted DNA strand 845 into a set of DNA fragments. The ligation key ID 840 specifies one or more ligation DNAzymes that may be used by the recipient 802 to ligate the set of DNA fragments in the proper order in order to recreate the encoded DNA strand 820. In some implementations, the sender 801 transmits the cleavage key ID 830 and the ligation key ID 840 via one or more computing devices and/or via one or more network connections or otherwise communicating the cleavage key ID 830 and the ligation key ID 840 to the recipient 802 or to a computing device accessible to the recipient 802. For example, the sender 801 transmits the cleavage key ID 830 and the ligation key ID 840 to the recipient 802 via email, text message, fax, voice message, video, messaging application, or other method of communication.

In some implementations, enzymes are used rather than DNAzymes. For example, anywhere cleavage DNAzymes are used, a CRISPR/Cas system may be used instead of or in addition to the cleavage DNAzymes. Anywhere ligation DNAzymes are used, a splint ligation process using T4 ligase may be used instead of or in addition to the ligation DNAzymes.

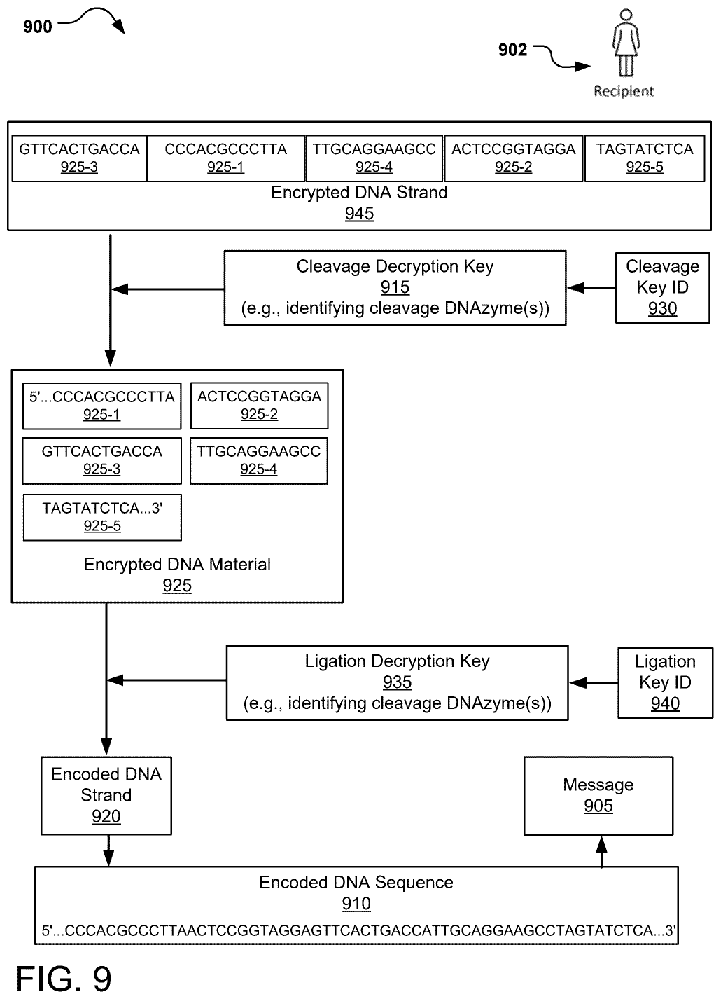

FIG. 9 illustrates an example 900 of decrypting an encrypted DNA strand 945 according to the encryption scheme described in FIG. 8 using a cleavage decryption key 915 and a ligation decryption key 935.

In some implementations, a recipient 902 receives the encrypted DNA strand 945 from a sender or otherwise accesses the encrypted DNA strand 945. In some implementations, the recipient 902 synthesizes the encrypted DNA strand 945 from a nucleotide sequence provided to the recipient 902 by the sender or otherwise accessed by the recipient 902.

The recipient 902 receives (e.g., from the sender) or otherwise accesses cleavage key ID 930 associated with a cleavage decryption key 915. The cleavage decryption key 915 specifies one or more cleavage DNAzymes for cleaving the encrypted DNA strand 945 into encrypted DNA material 925 that includes a set of fragments (e.g., fragment 925-1, fragment 925-2, fragment 925-3, fragment 925-4, fragment 925-5). The recipient 902 cleaves the encrypted DNA strand 945 into the encrypted DNA material 925 using the cleavage DNAzymes specified in the cleavage decryption key 915.

The recipient 902 receives (e.g., from the sender) or otherwise accesses ligation key ID 940 associated with a ligation decryption key 935. The ligation decryption key 935 specifies one or more ligation DNAzymes for ligating the set of fragments (encrypted DNA strand 945 into encrypted DNA material 925 that includes a set of fragments (e.g., fragment 925-1, fragment 925-2, fragment 925-3, fragment 925-4, fragment 925-5) of the encrypted DNA material 925 into an encoded DNA strand 920. The recipient 902 ligates the fragments of the encrypted DNA material 925 using the ligation DNAzymes specified in the ligation decryption key 935 to form the encoded DNA strand 920.

In some implementations, instead of conducting the cleavage reaction (e.g., using the cleavage decryption key) followed by the ligation reaction (e.g., using the ligation decryption key), the recipient 902 performs the cleavage reaction and the ligation reaction at the same time or at substantially the same time. For example, the recipient 902 cleaves the encrypted DNA strand 945 into the encrypted DNA material 925 and ligates the fragments of the encrypted DNA material 925 to form the encoded DNA strand 920 in a single decryption reaction.

As illustrated in FIG. 9, the order of the nucleotide sequence of the encrypted DNA strand 945 is different than the order of the nucleotide sequence of the encoded DNA strand 920 (e.g., which corresponds to the encoded DNA sequence 910). For example, the encrypted DNA strand 945 corresponds to the ligated fragments in the order of the fragment 925-3, followed by the fragment 925-1, followed by the fragment 925-4, followed by the fragment 925-2, followed by the fragment 925-5. However, the original encoded DNA strand 920, after ligation of the fragments of the encrypted DNA material 925, corresponds to the fragment 925-1, followed by the fragment 925-2, followed by the fragment 925-3, followed by the fragment 925-4, followed by the fragment 925-5.

The recipient 902 determines the encoded DNA sequence 910 of the encoded DNA strand 920, for example, using DNA sequencing techniques. For example, the encoded DNA sequence 910 includes the nucleotide sequence CCCACGCCCTTAACTCCGGTAGGAGTTCACTGACCATTGCAGGAAGCCTAGTATCTC A. The recipient 902 decodes the encoded DNA sequence 910 to determine the message 905 that was represented by the encrypted DNA strand 945. The message 905 may be one or more of a text, image data, video data, or other data. In some implementations, the recipient 902 may decode the message 905 according to a binary encoding scheme, a ternary encoding scheme, a symbol-linker encoding scheme (e.g., motif-by-motif encoding scheme), or other encoding scheme in which data (e.g., text, symbols, numbers, other values, etc.) of the message 905 is determined from the nucleotide sequence of the encoded DNA sequence 910.

Using the encryption scheme of FIG. 8 and the decryption scheme of FIG. 9, if an entity other than the recipient 902 obtains the encrypted DNA strand 945, the probability of guessing the correct cleavage sites and then ligating the cleaved fragments into the correct order of the fragments to reconstruct the encoded DNA sequence 910 is considerably low. Although the entity other than the recipient 902 may obtain some information by sequencing the encrypted DNA strand 945, the probability of guessing the correct message depends on both cleavage sites and order of cleaved fragments. If the length of the encrypted DNA strand 945 is n and the length of the DNA fragments of the encrypted DNA material 925 is x, the probability may be calculated by the following expression:

$${\sum }_{i = 1}^{n / x} \frac{1}{\frac{1}{n / x} \times ( i ) !} .$$

With an increase in the number of cleavage sites, it becomes increasingly difficult for the attacker to first guess the number of cleaved segments and then order them in the correct sequence. In such a scenario, the brute-force approach would be to break the encrypted DNA strand 945 into n fragments such that every fragment corresponds to one (1) nucleotide. In such a case, the probability of re-arranging the n fragments in the correct order would be 1/n!, where n represents the number of nucleotides that make up the sequence and not the number of fragments as in the encryption/decryption scheme of FIGS. 6-7. This is because the attacker does not know beforehand the number of segments the original sequence has been divided into. The recipient 902, though, has the information of the number of cuts made through the specially designed cleavage enzymes, which makes it easier to cut and re-order the segments in the correct sequence. Since the attacker does not have access to the cleavage keys, this type of segmenting and ordering is not possible and may be prohibitively expensive to perform. Like the encryption/decryption scheme of FIGS. 6-7, the there is still a possibility for a bad person to obtain information by sequencing the encrypted DNA strand 945. However, it would be exceedingly challenging to accurately cleave the encrypted DNA strand 945 into the correct fragments and then then ligate the cleaved short fragments in the correct order.

In some implementations, enzymes are used rather than DNAzymes. For example, anywhere cleavage DNAzymes are used, a CRISPR/Cas system may be used instead of or in addition to the cleavage DNAzymes. Anywhere ligation DNAzymes are used, a splint ligation process using T4 ligase may be used instead of or in addition to the ligation DNAzymes.

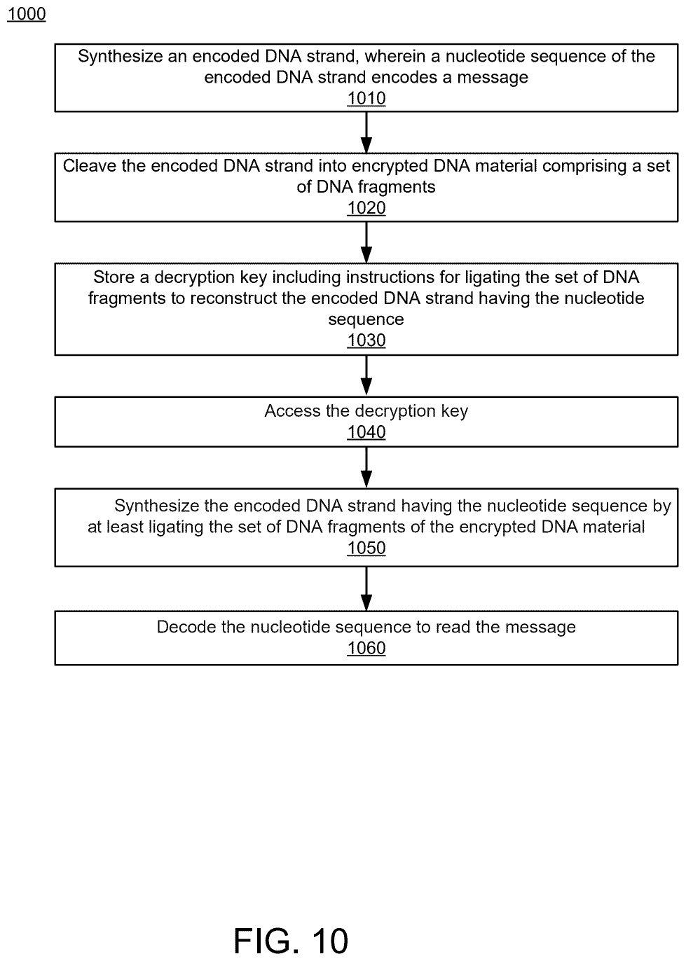

FIG. 10 depicts a process 1000 for encrypting DNA strands that encode messages. The example process 1000 includes operation 1010, operation 1020, operation 1030, operation 1040, and operation 1050.

Operation 1010 synthesizes an encoded DNA strand, wherein a nucleotide sequence of the encoded DNA strand encodes a message. In some implementations, the nucleotide sequence is an encrypted nucleotide sequence, wherein the encoded DNA strand encodes the encrypted nucleotide sequence.

In some implementations, encoding the message into the nucleotide sequence using one of a binary encoding scheme, a ternary encoding scheme, or a symbol-linker encoding scheme. Various encoding methods may be used in the described technology to encode a message into an encoded DNA sequence and to decode the encoded DNA sequence to read the message. For example, each nucleotide of the encoded DNA sequence may be assigned a bit pattern. In a one-to-one encoding method may represent each nucleotide as a single bit with a value of 0 or 1 (e.g., A, T=1, G, C=0). In a binary encoding method, each of the four possible nucleotides corresponds to a two-bit value, e.g., A=00, C=10, G=01, and T=11. In the binary encoding method, pairs of nucleotides may encode a corresponding binary pattern, as illustrated in Table 1 below:

TABLE 1

| DNA Oligo | Binary |
|---|---|
| AA | 0000 |
| AG | 0001 |
| AC | 0010 |
| AT | 0011 |
| GA | 0100 |
| GG | 0101 |
| GC | 0110 |
| GT | 0111 |
| CA | 1000 |
| CG | 1001 |
| CC | 1010 |
| CT | 1011 |
| TA | 1100 |
| TG | 1101 |
| TC | 1110 |
| TT | 1111 |

Using the example in Table 1 above, AA is 0000; the two base pair oligo stores 4 bits. As the oligo strand lengthens, more bits, bytes, and data can be stored. For example, an oligo that is 8 base pairs long stores 16 bits, or 2 bytes. It is noted that the example in Table 1 is an example of a primitive case and other bit mappings are possible where both the mapping and number of nucleotides per bit are different.

In a ternary encoding method, bits are converted to trits (e.g., ternary digits) and are represented by letters. For example, in the ternary encoding method, A may represent 0, G may represent 1, and T may represent 2. Using the following Table 2, the DNA strand can encode trits based on a value of a previous nucleotide in the sequence and a desired trit value:

TABLE 2

| Previous | 0 | 1 | 2 |
|---|---|---|---|
| T | A | C | G |
| G | T | A | C |
| C | G | T | A |
| A | C | G | T |

For example, when the previous nucleotide in the sequence is C, a following nucleotide G encodes a “0,” a following nucleotide T encodes a “1,” and a following nucleotide A encodes a “2.”

The previously discussed approaches (one-to-one, binary, ternary encoding methods) can either represent data in a bit-by-bit approach or may be combined with lookup tables. Methods other than the example methods described above may be used to encode data. For example, a symbol-linker (e.g., motif-by-motif) encoding approach may be used, which uses symbol portions and/or linker portions of the encoded DNA sequence and one or more lookup tables to determine values represented by particular configurations of symbols and/or linkers within the encoded DNA sequence. In some implementations, one or more supplemental DNA sequences encoding values may be used in addition to the encoded DNA sequence for use in a binary, ternary, symbol-linker, or other DNA encoding scheme.

Operation 1020 cleaves the encoded DNA strand into encrypted DNA material comprising a set of DNA fragments. Cleaving the encoded DNA stand into the encrypted DNA material includes cleaving the encoded DNA strand using one or more cleavage DNAzymes, each of the one or more cleavage DNAzymes configured to cleave the encoded DNA strand at a corresponding location along the nucleotide sequence.

Operation 1030 stores a decryption key including instructions for ligating the set of DNA fragments to reconstruct the encoded DNA strand having the nucleotide sequence. In some implementations, the decryption key includes a ligation key specifying one or more ligation DNAzymes for ligating the set of DNA fragments to reconstruct the encoded DNA strand. In some implementations, the decryption key is provided by a sender to a recipient. In some implementations, the decryption key includes a cleavage key specifying one or more cleavage DNAzymes for cleaving the encrypted DNA strand into the set of DNA fragments and a ligation key specifying one or more ligation DNAzymes for ligating the set of DNA fragments to reconstruct the encoded DNA strand. In some implementations, the decryption key further includes a nucleotide sequence decryption key for determining the nucleotide sequence from an encrypted nucleotide sequence represented by the encoded DNA strand.

Operation 1040 accesses the decryption key. In some implementations, the sender accesses the decryption key. In some implementations, the recipient accesses the decryption key or receives the decryption key from the sender. In some implementations, the sender provides the encrypted DNA material to the recipient. In some implementations, the sender provides nucleotide sequence information to the recipient for synthesizing the encrypted DNA material.

Operation 1050 synthesizes the encoded DNA strand having the nucleotide sequence by at least ligating the set of DNA fragments of the encrypted DNA material. Synthesizing the encoded DNA material may include ligating the set of DNA fragments of the encrypted DNA material into an encrypted DNA strand, wherein the nucleotide sequence of the encoded DNA strand is different from a nucleotide sequence of the encrypted DNA strand.

Operation 1060 decodes the nucleotide sequence to read the message.

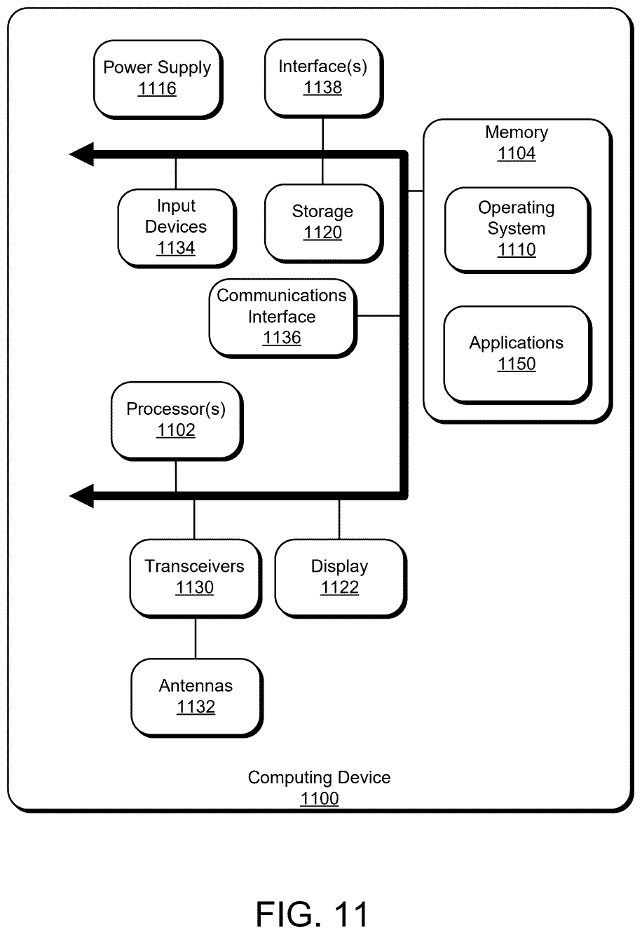

FIG. 11 illustrates an example computing device 1100 for use in implementing the described technology. The computing device 1100 may be a client computing device (such as a laptop computer, a desktop computer, or a tablet computer), a server/cloud computing device, an Internet-of-Things (IOT), any other type of computing device, or a combination of these options. The computing device 1100 includes one or more hardware processor(s) 1102 and a memory 1104. The memory 1104 generally includes both volatile memory (e.g., RAM) and nonvolatile memory (e.g., flash memory), although one or the other type of memory may be omitted. An operating system 1110 resides in the memory 1104 and is executed by the processor(s) 1102. In some implementations, the computing device 1100 includes and/or is communicatively coupled to storage 1120.

In the example computing device 1100, as shown in FIG. 11, one or more software modules, applications 1150, segments, and/or processors, such as a computing device of a sender and/or a recipient, an integrated circuit for performing one or more cleavage or ligation reactions described herein, and/or one or more components thereof. The storage 1120 may store one or more nucleotide sequences of encoded DNA strands, one or more nucleotide sequences of encrypted DNA strands, one or more nucleotide sequences of fragments of encrypted DNA material, one or more ligation key identifiers, one or more ligation keys, one or more cleavage key identifiers, one or more cleavage keys, one or more ligation encryption keys, one or more ligation decryption keys, one or more cleavage encryption keys, one or more cleavage decryption keys, one or more messages, one or more encoded DNA sequences, and other data and be local to the computing device 1100 or may be remote and communicatively connected to the computing device 1100.

The computing device 1100 includes a power supply 1116, which may include or be connected to one or more batteries or other power sources, and which provides power to other components of the computing device 1100. The power supply 1116 may also be connected to an external power source that overrides or recharges the built-in batteries or other power sources.

The computing device 1100 may include one or more communication transceivers 1130, which may be connected to one or more antenna(s) 1132 to provide network connectivity (e.g., mobile phone network, Wi-Fi®, Bluetooth®) to one or more other servers, client devices, IoT devices, and other computing and communications devices. The computing device 1100 may further include a communications interface 1136 (such as a network adapter or an I/O port, which are types of communication devices). The computing device 1100 may use the adapter and any other types of communication devices for establishing connections over a wide-area network (WAN) or local-area network (LAN). It should be appreciated that the network connections shown are exemplary and that other communications devices and means for establishing a communications link between the computing device 1100 and other devices may be used.

The computing device 1100 may include one or more input devices 1134 such that a user may enter commands and information (e.g., a keyboard, trackpad, or mouse). These and other input devices may be coupled to the server by one or more interfaces 1138, such as a serial port interface, parallel port, or universal serial bus (USB). The computing device 1100 may further include a display 1122, such as a touchscreen display.

The computing device 1100 may include a variety of tangible processor-readable storage media and intangible processor-readable communication signals. Tangible processor-readable storage can be embodied by any available media that can be accessed by the computing device 1100 and can include both volatile and nonvolatile storage media and removable and non-removable storage media. Tangible processor-readable storage media excludes intangible, transitory communications signals (such as signals per se) and includes volatile and nonvolatile, removable, and non-removable storage media implemented in any method, process, or technology for storage of information such as processor-readable instructions, data structures, program modules, or other data. Tangible processor-readable storage media includes, but is not limited to, RAM, ROM, EEPROM, flash memory or other memory technology, CDROM, digital versatile disks (DVD) or other optical disk storage, magnetic cassettes, magnetic tape, magnetic disk storage, or other magnetic storage devices, or any other tangible medium which can be used to store the desired information and which can be accessed by the computing device 1100. In contrast to tangible processor-readable storage media, intangible processor-readable communication signals may embody processor-readable instructions, data structures, program modules, or other data resident in a modulated data signal, such as a carrier wave or other signal transport mechanism. The term “modulated data signal” means a signal that has one or more of its characteristics set or changed in such a manner as to encode information in the signal. By way of example, and not limitation, intangible communication signals include signals traveling through wired media such as a wired network or direct-wired connection, and wireless media such as acoustic, RF, infrared, and other wireless media.

The above specification and examples provide a complete description of the structure and use of exemplary implementations of the invention. The above description provides specific implementations. It is noted that although not specifically stated, between any of the assembly steps described throughout this description, any additional steps may be added as needed or desired, for example, a PCR amplification step, a purification step, or both. Either of these steps could be performed after a synthesis step (e.g., Gibson assembly step or other synthesis method or protocol). It is to be understood that other implementations are contemplated and may be made without departing from the scope or spirit of the present disclosure. The above-detailed description, therefore, is not to be taken in a limiting sense. While the present disclosure is not so limited, an appreciation of various aspects of the disclosure will be gained through a discussion of the examples provided.

Unless otherwise indicated, all numbers expressing feature sizes, amounts, and physical properties are to be understood as being modified by the term “about,” whether or not the term “about” is immediately present. Accordingly, unless indicated to the contrary, the numerical parameters set forth are approximations that can vary depending upon the desired properties sought to be obtained by those skilled in the art utilizing the teachings disclosed herein.

As used herein, the singular forms “a,” “an,” and “the” encompass implementations having plural referents, unless the content clearly dictates otherwise. As used in this specification and the appended claims, the term “or” is generally employed in its sense including “and/or” unless the content clearly dictates otherwise.

Spatially related terms, including but not limited to, “bottom,” “lower”, “top”, “upper”, “beneath”, “below”, “above”, “on top”, “on,” etc., if used herein, are utilized for ease of description to describe spatial relationships of an element(s) to another. Such spatially related terms encompass different orientations of the device in addition to the particular orientations depicted in the figures and described herein. For example, if a structure depicted in the figures is turned over or flipped over, portions previously described as below or beneath other elements would then be above or over those other elements.

Since many implementations of the invention can be made without departing from the spirit and scope of the invention, the invention resides in the claims hereinafter appended. Furthermore, structural features of the different implementations may be combined in yet another implementation without departing from the recited claims.

What is claimed is:

1. A method, comprising:
providing, to a recipient, encrypted DNA material comprising a set of DNA fragments; and
providing, to the recipient, a decryption key including instructions for ligating the set of DNA fragments to construct an encoded DNA strand having a nucleotide sequence encoding a message.

2. The method of claim 1, further comprising: synthesizing the encoded DNA strand; and wherein providing the encrypted DNA material to the recipient includes:
cleaving the encoded DNA strand into the encrypted DNA material; and
providing, to the recipient, the set of DNA fragments cleaved from the encoded DNA strand

3. The method of claim 2, wherein cleaving the encoded DNA strand into the encrypted DNA material includes cleaving the encoded DNA strand using one or more cleavage DNAzymes, each of the one or more cleavage DNAzymes configured to cleave the encoded DNA strand at a corresponding location along the nucleotide sequence.

4. The method of claim 2, wherein cleaving the encoded DNA strand into the encrypted DNA material includes cleaving the encoded DNA strand using one or more cleavage enzymes, each of the one or more cleavage enzymes configured to cleave the encoded DNA strand at a corresponding location along the nucleotide sequence.

5. The method of claim 1, wherein the decryption key includes a ligation key specifying one or more ligation enzymes for ligating the set of DNA fragments to reconstruct the encoded DNA strand.

6. The method of claim 1, wherein the decryption key includes a ligation key specifying one or more ligation DNAzymes for ligating the set of DNA fragments to reconstruct the encoded DNA strand.

7. The method of claim 1, further comprising ligating the set of DNA fragments into an encrypted DNA strand, wherein the nucleotide sequence of the encoded DNA strand is different from a nucleotide sequence of the encrypted DNA strand.

8. The method of claim 7, wherein the decryption key includes a cleavage key specifying one or more cleavage DNAzymes or cleavage enzymes for cleaving the encrypted DNA strand into the set of DNA fragments and a ligation key specifying one or more ligation DNAzymes or ligation enzymes for ligating the set of DNA fragments to reconstruct the encoded DNA strand.

9. The method of claim 1, further comprising:
prior to synthesizing the encoded DNA strand, encrypting the nucleotide sequence encoding the message into an encrypted nucleotide sequence, wherein the encoded DNA strand encodes the encrypted nucleotide sequence, the encoded DNA strand including the encrypted nucleotide sequence, the decryption key further including a decryption key for determining the nucleotide sequence from the encrypted nucleotide sequence.

10. The method of claim 1, wherein providing the encrypted material to the recipient includes providing nucleotide sequence information to the recipient for synthesizing the encrypted DNA material.

11. The method of claim 1, further comprising encoding the message into the nucleotide sequence using one of a binary encoding scheme, a ternary encoding scheme, or a symbol-linker encoding scheme.

12. A method, comprising:
synthesizing an encoded DNA strand having a nucleotide sequence that encodes a message by at least ligating a set of DNA fragments of encrypted DNA material; and
decoding the nucleotide sequence to determine the message.

13. The method of claim 12, wherein ligating the set of DNA fragments of the encrypted DNA material includes ligating the set of DNA fragments using one or more ligation DNAzymes or ligation enzymes, each of the one or more ligation DNAzymes or ligation enzymes configured to ligate a corresponding first DNA fragment of the set of DNA fragments to a corresponding second DNA fragment of the set of DNA fragments.

14. The method of claim 12, further comprising accessing a decryption key, wherein the decryption key includes a ligation key specifying one or more ligation DNAzymes or ligation enzymes for ligating the set of DNA fragments to reconstruct the encoded DNA strand.

15. The method of claim 12, further comprising cleaving an encrypted DNA strand into the encrypted DNA material, wherein the nucleotide sequence of the encoded DNA strand is different from a nucleotide sequence of the encrypted DNA strand.

16. The method of claim 15 further comprising accessing a cleavage decryption key specifying one or more cleavage DNAzymes or cleavage enzymes, wherein cleaving the encrypted DNA strand into the encrypted DNA material includes cleaving the encrypted DNA strand using the one or more cleavage DNAzymes or cleavage enzymes.

17. The method of claim 10, wherein the encoded DNA strand encodes an encrypted nucleotide sequence, the encoded DNA strand including the encrypted nucleotide sequence, and further comprising:
accessing a decryption key; and
determining the nucleotide sequence by decrypting the encrypted nucleotide sequence.

18. The method of claim 12, further comprising:
accessing nucleotide sequence information for synthesizing the encrypted DNA material; and
synthesizing, based on the nucleotide sequence information, the encrypted DNA material.

19. A method, comprising:
synthesizing an encoded DNA strand, wherein a nucleotide sequence of the encoded DNA strand encodes a message;
cleaving the encoded DNA strand into encrypted DNA material comprising a set of DNA fragments;
storing a decryption key including instructions for ligating the set of DNA fragments to reconstruct the encoded DNA strand having the nucleotide sequence;
accessing the decryption key;
synthesizing the encoded DNA strand having the nucleotide sequence by at least ligating the set of DNA fragments of the encrypted DNA material; and
decoding the nucleotide sequence to read the message.

20. The method of claim 19,
wherein cleaving the encoded DNA strand into the encrypted DNA material includes cleaving the encoded DNA strand using one or more cleavage DNAzymes or cleavage enzymes, each of the one or more cleavage DNAzymes or cleavage enzymes configured to cleave the encoded DNA strand at a corresponding location along the nucleotide sequence,
wherein ligating the set of DNA fragments of the encrypted DNA material includes ligating the set of DNA fragments using one or more ligation DNAzymes or ligation enzymes, each of the one or more ligation DNAzymes or ligation enzymes configured to ligate a corresponding first DNA fragment of the set of DNA fragments to a corresponding second DNA fragment of the set of DNA fragments.
